(19) United States
(12) Patent Application Publication
(10) Pub. No.: US 2021/0082570 A1
(43) Pub. Date: Mar. 18, 2021

(54) ARTIFICIAL INTELLIGENCE (AI) POWERED ANALYSIS OF OBJECTS OBSERVABLE THROUGH A MICROSCOPE

(71) Applicant: Celly.AI, Walnut, CA (US)

(72) Inventor: Ansar S. Zhalyalov, Moscow (RU)

(21) Appl. No.: 17/011,789

(22) Filed: Sep. 3, 2020

Related U.S. Application Data
(60) Provisional application No. 62/899,918, filed on Sep. 13, 2019.

(51) Int. Cl. G16H 30/40; G16H 50/20; G16H 30/20; G06T 7/00; A61M 1/36; G06F 16/58

(52) U.S. Cl. CPC G16H 30/40; G16H 50/20; G06F 16/58; G06T 7/0012; A61M 1/3607; G16H 30/20

(57) ABSTRACT

Introduced here are computer programs and associated computer-implemented techniques for autonomously analyzing images of blood smears that are captured during an examination session. Initially, an optical adapter device may be attached to the eyepiece of a microscope. The optical adapter device may be designed to facilitate alignment of the camera of an electronic device with the eyepiece of the microscope. As part of an examination session, the electronic device may generate a series of images of a blood smear on a slide that is viewable through the eyepiece. Generally, the electronic device is detachably connectable to the optical adapter so that it can be removed from the optical adapter device after generating the series of images. The series of images can be partially or entirely processed by a diagnostic platform in an automated manner using artificial intelligence or machine learning algorithms in order to streamline the diagnostic process.

### CROSS-REFERENCE TO RELATED APPLICATIONS

This application claims priority to U.S. Provisional Application No. 62/899,918, filed on Sep. 13, 2019, which is incorporated herein by reference in its entirety,

### TECHNICAL FIELD

Various embodiments concern computer programs and associated computer-implemented techniques for analyzing images captured during microscopic examination.

### BACKGROUND

A blood film—or peripheral blood smear—is a thin layer of blood that has been smeared on a slide and then stained so that the various blood cells can be examined microscopically. Blood films are examined in the investigation of hematological disorders. For instance, blood films are routinely employed to look for abnormal cells and parasites. Accordingly, blood films may be used to detect a wide range of disorders, including anemia, infection, malaria, and leukemia.

Blood films are made by placing a drop of blood on one end of a slide and then using a spreader slide to disperse the blood over the length of the slide. The goal is to create a region (referred to as a “monolayer”) where the cells are spaced far enough apart to be counted, differentiated, etc. Generally, the monolayer is found in the “feathered edge” created by the spreader slide as it draws the blood across the slide.

The slide can then be left to dry, and the blood may be fixed to the slide by immersing it in a fixative such as methanol. The fixative may be necessary for high-quality presentation of cellular detail. After fixation, the slide can be stained to distinguish the cells from one another. Routine analysis is usually performed on blood films stained to allow for the detection of white blood cells, red blood cells, and/or platelet abnormalities. Specialized stains may aid in the differential diagnosis of blood disorders. The monolayer can be viewed under a microscope by a trained specialist after staining is completed. Examples of trained specialists include pathologists and laboratory scientists.

### BRIEF DESCRIPTION OF THE DRAWINGS

FIG. 1 illustrates a network environment that includes a microscope, an optical adapter, an electronic device, an object detection module, a computer server, an object classification module, and a display.

FIG. 2A illustrates how optical adapters may be connected to the eyepieces of microscopes.

FIG. 2B depicts another example of an optical adapter that can be installed on the eyepiece of a microscope

FIG. 3 depicts how a separate holder that is connected to an electronic device can be detachably connected to an optical adapter secured to the eyepiece of a microscope.

FIG. 4 illustrates a network environment that includes a diagnostic platform.

FIG. 5 illustrates an example of an electronic device on which a diagnostic platform may reside.

FIG. 6A depicts an example of interface in which the cells deemed abnormal in a blood smear have been visually outlined and labeled.

FIG. 6B depicts an example of an interface in which the results of an analysis have been presented.

FIG. 6C illustrates how a series of images, rather than a composite image of the entire slide, can be processed by a diagnostic platform and then presented in a report.

FIG. 7 depicts a flow diagram of a process for analyzing images of cells that are observable through a microscope.

FIG. 8 depicts a flow diagram of a process for inferring health based on automated analysis of images of a blood smear.

FIG. 9 is a block diagram illustrating an example of a processing system in which at least some operations described herein can be implemented.

Various features of the technologies described herein will become more apparent to those skilled in the art from a study of the Detailed Description in conjunction with the drawings. Embodiments are illustrated by way of example and not limitation in the drawings, in which like references may indicate similar elements. While the drawings depict various embodiments for the purpose of illustration, those skilled in the art will recognize that alternative embodiments may be employed without departing from the principles of the technologies. Accordingly, while specific embodiments are shown in the drawings, the technology is amenable to various modifications.

### DETAILED DESCRIPTION

A blood film—or peripheral blood smear—is a thin layer of blood that has been smeared on a slide and then stained so that the various blood cells can be examined microscopically. Blood films are made by placing a drop of blood on one end of a slide and then using a spreader slide to disperse the blood over the length of the slide. The goal is to create a region (referred to as a “monolayer”) where the cells are spaced far enough apart to be counted and differentiated. Generally, the slide is stained to distinguish the cells from one another, and the monolayer is viewed under a microscope after staining is completed.

Traditionally, a trained specialist is responsible for manually examining the monolayer in order to characterize and then record the morphology of individual cells in the blood. However, such an approach is prone to several problems that affect the accuracy and speed of pathological characterization. First, this process is susceptible to errors due to fatigue, inattention, subjectivity, and the like. For example, overlapping cells may be miscounted, and normal cells may be mistaken for abnormal cells (or vice versa) since small variations in the shape, size, or color of cells tend to be largely imperceptible—even at magnification levels up to 1000×. Second, this process is time consuming—even for trained specialists—which not only increases the costs of pathological characterization but may result in delays (e.g., in treatment) that have significant ramifications. And third, the salaries of trained specialists, such as pathologists, are much higher than individuals with less training, such as laboratory technicians.

Introduced here, therefore, are computer programs and associated computer-implemented techniques for autonomously analyzing images of blood smears that are captured during an examination session. Initially, an optical adapter device (or simply “optical adapter”) may be attached to the eyepiece of a microscope. As further discussed below, the optical adapter may be designed to facilitate alignment of the camera of an electronic device with the eyepiece of the microscope. As part of an examination session, the electronic device may generate a series of images of a blood smear on a slide that is viewable through the eyepiece. Generally, the electronic device is detachably connectable to the optical adapter. Thus, the electronic device may be secured to the optical adapter and then removed from the optical adapter after generating the series of images.

The series of images can then be processed by a diagnostic platform in order to streamline the examination session. For the purpose of illustration, the diagnostic platform may be described as a computer program that autonomously performs aspects of microscopic examination. However, elements of the diagnostic platform may be embodied in software, firmware, or hardware. Moreover, those skilled in the art will recognize that the diagnostic platform need not necessarily be executed by the electronic device that generates the series of images. For example, the diagnostic platform may be described as a mobile application on a mobile phone that can be detachably secured to the eyepiece of a microscope. However, in some embodiments, the diagnostic platform is hosted on an electronic device (e.g., a computer server) that is communicatively connected to the mobile phone.

An individual may be able to view these images through the diagnostic platform by activating an image analysis mode. In this mode, the diagnostic platform can (i) continuously calculate shift in the field of view; (ii) detect, classify, and count objects in the images using, for example, artificial intelligence (AI) algorithms; (iii) send the images to another electronic device, such as a computer server, for analysis, reporting, etc. and/or (iv) create a digital copy of the slide by stitching the images together; Since the images may overlap to some degree, this stitching must be done with caution to ensure that portions of the slide are not missed (e.g., due to improper/imprecise overlapping of images). In some embodiments, the images received by the electronic device are accompanied by location identifiers. Examples of location identifiers include Global Positioning System (GPS) coordinates and wireless access point (WAP) identifiers. These location identifiers may be used to help identify the individual whose blood is being imaged, the enterprise responsible for managing the examination session, or simply to ensure that images generated during an examination session are properly associated with one another.

The technologies described herein can be employed as part of a cost- and resource-effective approach to analyzing blood smears by automating aspects of microscopy testing. For example, the diagnostic platform can create a scan of an entire slide by combining a series of images generated over an interval of time, differentiate objects detected in the scan, and then identify objects representative of abnormalities through the use of artificial intelligence. Such an approach allows tasks that have historically been performed by trained specialists to be reassigned to less-qualified (and thus less-expensive) individuals.

Embodiments may be described in the context of blood smear analysis or complete blood cell count test. However, the technologies described herein could be used for various kinds of microscopy tests, including those that are suitable for partial or entire automation. Examples of such microscopy tests include those related to microbiology, urine analysis, stool analysis, semen analysis, cytology analysis (body fluids), histopathology analysis (biopsy), or veterinary analysis.

While embodiments may be described in the context of computer-executable instructions, aspects of the technologies described herein can be implemented via hardware, firmware, or software. As an example, a diagnostic platform embodied as a software program executing on an electronic device (e.g., a mobile phone) may be able to examine multiple images of a blood smear that are generated as part of an examination session, apply an object detection algorithm to detect the cells within those images, and then transmit those images (e.g., with bounding boxes identifying the detected cells) to a destination for classification of the cells. The destination could be, for example, a computer server that is communicatively connected to the electronic device across a network. As another example, a diagnostic platform may be able to examine multiple images of a blood smear that are generated as part of an examination session, stitch those images together to form a slide scan, identify objects by applying an object detection algorithm to the slide scan, classify each object identified in the slide scan, and then produce an output indicative of a diagnosis for a given ailment based on the classified objects.

### Terminology

References in this description to “an embodiment” or “one embodiment” means that the particular feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the words “comprise” and “comprising” are to be construed in an inclusive sense rather than an exclusive or exhaustive sense (i.e., in the sense of “including but not limited to”). The term “based on” is also to be construed in an inclusive sense rather than an exclusive or exhaustive sense. Thus, unless otherwise noted, the term “based on” is intended to mean “based at least in part on.”

The terms “connected,” “coupled,” or any variant thereof is intended to include any connection or coupling between two or more elements, either direct or indirect. The connection/coupling can be physical, logical, or a combination thereof. For example, objects may be electrically or communicatively coupled to one another despite not sharing a physical connection.

The term “module” refers broadly to software components, firmware components, and/or hardware components. Modules are typically functional components that generate output(s) based on specified input(s). A computer program may include one or more modules. Thus, a computer program may include multiple modules responsible for completing different tasks or a single module responsible for completing all tasks.

When used in reference to a list of multiple items, the term “or” is intended to cover all of the following interpretations: any of the items in the list, all of the items in the list, and any combination of items in the list.

The sequences of steps performed in any of the processes described here are exemplary. However, unless contrary to physical possibility, the steps may be performed in various sequences and combinations. For example, steps could be added to, or removed from, the processes described here. Similarly, steps could be replaced or reordered. Thus, descriptions of any processes are intended to be open-ended. Moreover, some or all of the steps of these processes could be performed by any combination of one or more computing devices that collectively or individually implement aspects of a multi-dimensional program described herein.

### Technology Overview

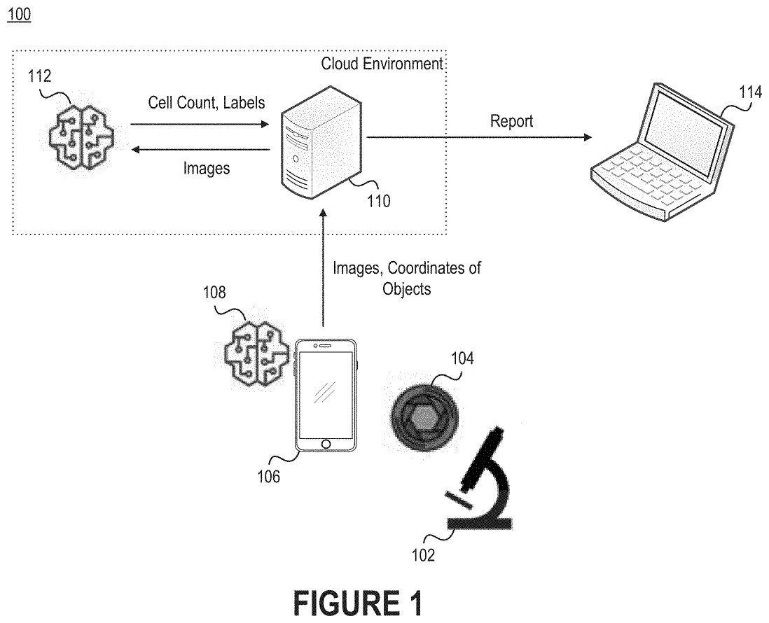

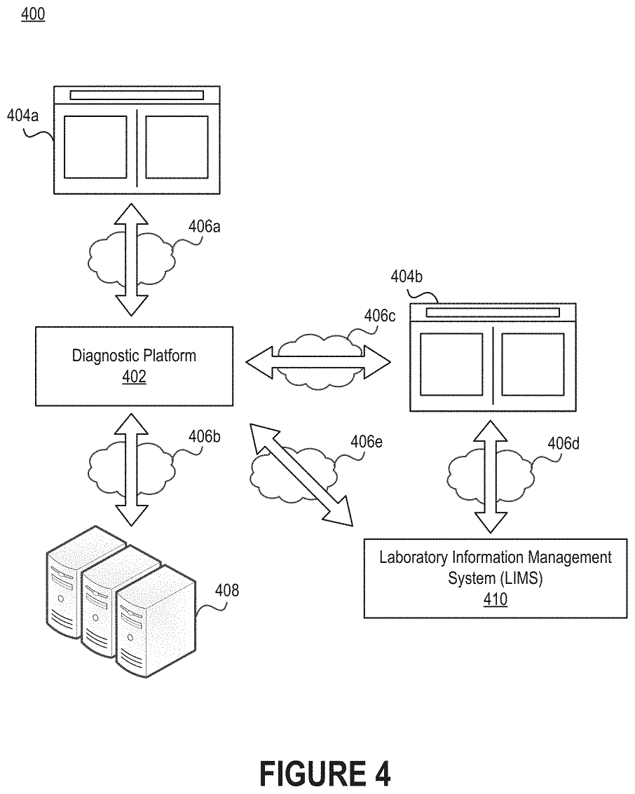

FIG. 1 illustrates a network environment 100 that includes a microscope 102, an optical adapter 104, an electronic device 106, an object detection module 108, a computer server 110, an object classification module 112, and a display 114. Note that embodiments of the network environment 100 include some or all of these items. For example, some embodiments include a single object detection module that is part of a diagnostic platform, as further discussed below with reference to FIGS. 4-8. In such embodiments, the object classification module may be absent. Similarly, the display 114 may be absent if results are displayed by the electronic device 106. In the embodiment shown in FIG. 1, the object detection module 108 executes on the electronic device 106 that is responsible for generating images of the slide positioned in the microscope 102, Accordingly, images generated by the electronic device 106 may be examined locally (e.g., by object detection module 108) and/or remotely (e.g., by object classification module 112).

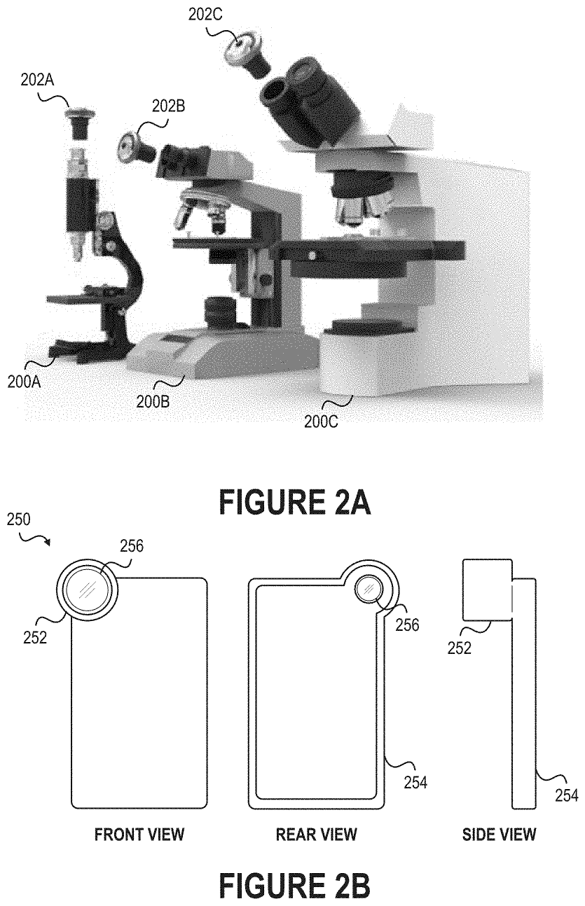

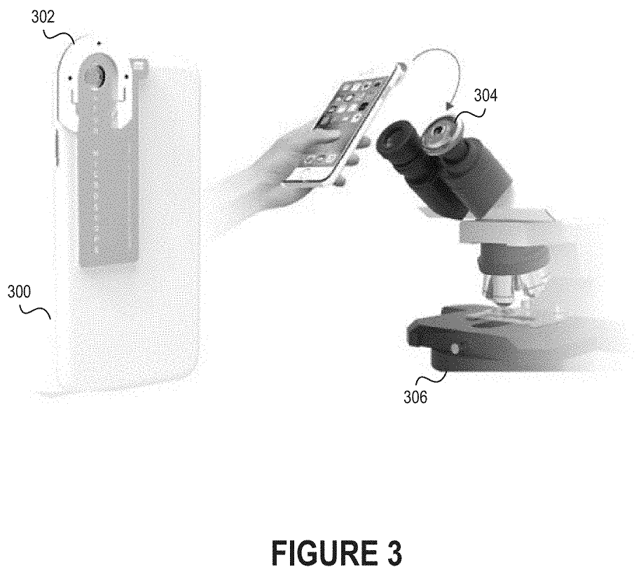

The optical adapter 104 is designed to be physically connected to the eyepiece of the microscope 102, as further discussed below with respect to FIGS. 2-3. The optical adapter 104 may be detachably connected to the eyepiece of the microscope 102, or the optical adapter 104 may be fixedly secured to the eyepiece of the microscope 102. Thus, the optical adapter 104 may be readily removable from the eyepiece of the microscope 102 in some embodiments and not readily removable from the eyepiece of the microscope 102 in other embodiments.

The electronic device 106, meanwhile, can be detectably connected to the optical adapter 104. In FIG. 1, the electronic device 106 is a mobile phone. However, the electronic device 106 could be a laptop computer, mobile workstation, etc. The optical adapter 104 may be designed so that when the electronic device 106 is secured thereto, the camera of the electronic device 106 is aligned with the eyepiece of the microscope 102. Over the course of a microscopic examination session (or simply “examination session”), the electronic device 106 can generate a series of images of a blood smear on a slide that is observable through the eyepiece of the microscope 102. In some embodiments the series of images is representative of a video feed, while in other embodiments the series of images is representative of static images captured on a periodic basis. For instance, the series of images may be generated at a predetermined frequency (e.g., every 2 or 3 seconds), or each of the series of images may be generated responsive to receiving input indicative of a request to capture the image. As an example, an individual may be responsible for relocating the slide across the stage. Each time that the individual relocates the slide (e.g., by shifting the slide in one direction by several millimeters), the individual may provide input that an image should be generated. For instance, the individual might interact with a digital element shown on the display of the electronic device 106, or the individual might interact with a physical button of the electronic device 106.

These images can be provided to the object detection module 108 for further processing. In some embodiments images are streamed to the object detection module 108 in real time as those images are generated, while in other embodiments images are forwarded to the object detection module 108 at a later point in time. For example, images may not be provided to the object detection module 108 until the entire series of images has been generated.

The object detection module 108 may employ AI-driven algorithms to detect objects within these images. Examples of such algorithms include Region-CNN (R-CNN), Fast R-CNN, You Only Look Once (YOLO), and Single-Shot MultiBox Detector (SSD). Accordingly, the object detection module 108 may identify each object in each image. Moreover, the object detection module 108 may establish a count of the total number of objects in a given image, identify the segment of the slide captured in each image, etc. For example, the object detection module 108 executing on the mobile phone may employ SSD, while the object classification module 112 executing on the computer server 110 may employ a convolutional neural network such as VGG Net designed for image classification, etc.

Information gleaned from the images by the object detection module 108 may be provided to the object classification module 112 executing on the computer server 110 in the form of metadata. For example, the object detection module 108 may transmit the series of images and accompanying metadata to the object classification module 112 for further analysis. The accompanying metadata may include labels for the objects, coordinates, identifiers for any objects determined to be abnormal, etc. In some embodiments, the object detection module 108 may perform deduplication so that only one image of multiple adjacent images is transmitted to the object classification module 112. Thus, a subset of all images generated by the electronic device 106 or images of detected objects may be sent to the object classification module 112 for further analysis. In some embodiments, images, classifications, features, or bounding boxes determined by the object detection module 108 are not transmitted to the object classification module 112

Much like the object detection module 108, the object classification module 112 may employ AI-driven algorithms to classify objects in the images uploaded by the electronic device 106. For example, in embodiments where the object detection module 108 is designed to process images in near real time, the object classification module 112 may employ more time- and resource-intensive AI-driven algorithms.

Generally, the object detection and classification modules 108, 112 is designed to produce a count of all objects in the images generated during an examination session and their classes. For example, each cell may be labeled as either normal or abnormal based on its shape, size, and/or color.

In some embodiments, the computer server is designed to produce a report that identifies the cells deemed to be abnormal or unhealthy, a proposed diagnosis (e.g., based on analysis of the cells determined to be present), the total number of cells, or other information. The report may be transmitted to a display 114 for review by an individual. The individual may be, for example, a medical professional (e.g., a physician or specialist) responsible for rendering a diagnosis and prescribing appropriate treatment to the patient (also referred to as the “subject”) from whom the blood was drawn. Note, however, that the report could also be presented on the display of the electronic device 106. Thus, a separate display 114 may not be present in some embodiments.

In FIG. 1, the object detection and classification modules 108, 112 are shown as separate computer programs that are executing on different devices. However, these programs may be part of the same diagnostic platform that is responsible for analyzing images of the blood smear that is observable through the eyepiece of the microscope 102. The diagnostic platform is described in greater depth below with reference to FIGS. 4-6.

### Overview of Optical Adapters

FIG. 2A illustrates how optical adapters 202A-C may be connected to the eyepieces of microscopes 200A-C. As noted above, the optical adapters 202A-C are normally detachable connectable to the microscopes 200A-C. For example, the optical adapters 202A-C may have structural features designed to mate or complement structural features of the microscopes 200A-C, as further discussed with reference to FIG. 2B. Alternatively, the optical adapters 202A-C could be fixedly secured to the microscopes 200A-C. For example, the optical adapters 202A-C could be connected to the microscopes 200A-C using permanent or semi-permanent means, such as adhesives or mechanical fasteners (e.g., screws, bolts, brackets, etc.). Note that while an optical adapter may be described as being “connected to” or “installed on” the eyepiece of a microscope, the optical adapter need not necessarily be physically connected to the eyepiece itself. For example, an optical adapter may be designed to be installed on a component proximate to the eyepiece, such as the arm or tube leading to the eyepiece, so that the optical adapter is aligned with the eyepiece.

FIG. 2B depicts another example of an optical adapter 250 that can be installed on the eyepiece of a microscope. As shown in FIG. 2B, the optical adapter 250 may have an elongated structural feature 252. The optical adapter 250 can be readily installed on the microscope by simply slidably engaging the elongated structural feature 252 with the eyepiece. Generally, the elongated structural feature 252 is designed such that the optical adapter 250 is compatible with microscopes produced by Nikon, Olympus, Zeiss, and other manufacturers.

In some embodiments, one or more glass lenses 256 are located within the elongated structural feature 252. These glass lens(es) may further magnify whatever is viewable through the eyepiece of the microscope. For example, these glass lens(es) may provide 2.5×, 5×, or 10× magnification. In other embodiments, the elongated structural feature 252 is complete devoid of any items. For example, the elongated structural feature 252 may be a vacant cylinder designed to facilitate alignment.

Moreover, the optical adapter 250 may include a securement feature for securely retaining an electronic device. The securement feature may be designed to accommodate certain types of electronic devices. Here, for example, the optical adapter 250 includes a peripheral frame 254 designed to accommodate a mobile phone. Accordingly, an individual may connect a mobile phone to the optical adapter 250 by installing the mobile phone within the peripheral frame 254 so that its camera is aligned with the elongated structural feature 252. Other examples of securement features include adhesive films, magnets, and mechanical features such as clips, brackets, and the like.

FIG. 3 depicts how a separate holder 302 that is connected to an electronic device 300 can be detachably connected to an optical adapter 304 secured to the eyepiece of a microscope 306. The holder 302 may be, for example, a rigid component that facilitates coupling of the electronic device 300 to the optical adapter 304. Moreover, the holder 302 may facilitate alignment of the camera of the electronic device 300 with the glass lens(es), if any, of the optical adapter 304. The holder 302 may be comprised of plastic, metal, or another rigid material. Those skilled in the art will recognize that the electronic device 300, holder 302, optical adapter 304, and microscope 306 can be connected to one another in various orders.

Holders may be useful in scenarios where different types of electronic devices will be used with a single microscope. Assume, for example, that the microscope includes an optical adapter as shown in FIG. 3. In such a scenario, a first individual with a first type of electronic device (e.g., an Apple iPhone mobile phone) may obtain an appropriate holder and then secure her electronic device to the optical adapter. Thereafter, a second individual with a second type of electronic device (e.g., a Samsung Galaxy mobile phone) wishes to connect her phone to the optical adapter. Rather than replace the optical adapter, the second individual can instead obtain an appropriate holder and then secure her electronic device to the optical adapter.

### Overview of Diagnostic Platform

FIG. 4 illustrates a network environment 400 that includes a diagnostic platform 402. Individuals can interface with the diagnostic platform 402 via an interface 404a. Similarly, individuals can interface with a laboratory information management system (LIMS) 410 via an interface 404b. The diagnostic platform 402 may be responsible for examining one or more images generated by an electronic device of content viewable through the eyepiece of a microscope, detecting objects in those image(s), classifying the objects, and then generating a proposed diagnosis for one or more ailments based on the classified objects. The diagnostic platform 402 may also be responsible for creating interfaces through which individuals can perform tasks such as view images (or altered images with, for example, the classified objects accompanied by labels), label objects, and manage preferences.

As discussed above, the images examined by the diagnostic platform 402 may be generated by an electronic device that is connected to the microscope using an optical adapter. For example, a mobile phone may be detachably connected to the optical adapter in order to generate a series of images of a blood smear that is observable through the eyepiece of the microscope. In some embodiments, the electronic device (or, more specifically, a computer program executing on the electronic device) is configured to automatically upload the series of images to the diagnostic platform 402. The computer program may be the native camera application executing on the electronic device. In other embodiments, an individual is responsible for manually uploading the series of images through the interface 404a that is accessible on the electronic device. For example, an individual may be permitted to browse a storage medium (e.g., the camera roll on the electronic device) and select those images that should be provided for analysis.

The images could be associated with the individual who accesses the interfaces 404a-b or some other person. For example, the interfaces 404a-b may enable a person to view information related to her own physiological state, such as proposed diagnoses for different ailments. As another example, the interfaces 404a-b may enable an individual to view information related to the physiological state of some other person. The individual may be a medical professional (e.g., a physician or a nurse) responsible for monitoring, managing, or treating the other person. Some interfaces are configured to facilitate interactions between patients (also referred to as “subjects”) and medical professionals, while other interfaces are configured to serve as informative dashboards for either patients or medical professionals.

As noted above, the diagnostic platform 402 may reside in a network environment 400. Thus, the diagnostic platform 402 may be connected to one or more networks 406a-c. Moreover, as shown in FIG. 4, the diagnostic platform 402 may be connected to the LIMS 410 either directly, via a network 406e, or indirectly, via one or more networks 406c-d. The networks 406a-e can include personal area networks (PANs), local area networks (LANs), wide area networks (WANs), metropolitan area networks (MANs), cellular networks, the Internet, etc. Additionally or alternatively, the diagnostic platform 402 can be communicatively coupled to electronic device(s) over a short-range communication protocol, such as Bluetooth® or Near Field Communication (NFC). By interfacing with the LIMS 410, the diagnostic platform 402 may be able to obtain, infer, or derive information regarding the individual whose blood is being analyzed. For example, the diagnostic platform 402 may obtain information regarding the individual to build a patient profile in which results of the analysis can be stored, or the diagnostic platform 402 may obtain information regarding the individual so that results can be sent to the LIMS 410 for inclusion in a patient profile.

The interfaces 404a-b may be accessible via a web browser, desktop application, mobile application, or over-the-top (OTT) application. For example, an individual may be able to access interfaces through which she can review outputs produced by the diagnostic platform 402 via a mobile application executing on a mobile phone or tablet computer that generated the images being analyzed. As another example, an individual may be able to access interfaces through which she can review outputs produced by the diagnostic platform 402 via a web browser executing on a personal computer. In such embodiments, the personal computer may be communicatively connected to a network-accessible server system 408 on which the diagnostic platform 402 is hosted. Accordingly, the interfaces 404a-b may be viewed on personal computers, tablet computers, mobile phones, wearable electronic devices (e.g., watches or fitness accessories), network-connected (“smart”) electronic devices (e.g., televisions or home assistant devices), gaming consoles, virtual/augmented reality systems (e.g., head-mounted displays), or another type of electronic device.

As further discussed below, components of the diagnostic platform 402 may be hosted locally in some embodiments. That is, at least part of the diagnostic platform 402 may reside on the electronic device used to access the interfaces 404a-b. For example, the diagnostic platform 402 may be embodied as a mobile application executing on a mobile phone that is responsible for generating the images of the slide that is viewable through the microscope.

In other embodiments, the diagnostic platform 402 is entirely executed by a cloud computing service operated by, for example, Amazon Web Services® (AWS), Google Cloud Platform™, or Microsoft Azure®. In such embodiments, the diagnostic platform 402 may reside on a network-accessible server system 408 comprised of one or more computer servers. These computer server(s) can include images, models for analyzing the images such as neural networks, patient information (e.g., profiles, credentials, and health-related information such as age, disease classification, etc.), and other assets.

Those skilled in the art will recognize that this information could also be distributed amongst a network-accessible server system and one or more electronic devices. For example, some data (e.g., images of blood smears) may be stored on, and processed by, a personal electronic device for security and privacy purposes. This data may be processed (e.g., obfuscated or anonymized) before being transmitted to the network-accessible server system 408. As another example, modules for processing data may be located in several locations to achieve timeliness and thoroughness. For instance, a first module for processing the data may be hosted on the electronic device used to generate the images while a second module for processing the data may be hosted on the network-accessible server system 408. Such a configuration is described above with reference to object detection modules in FIG. 1.

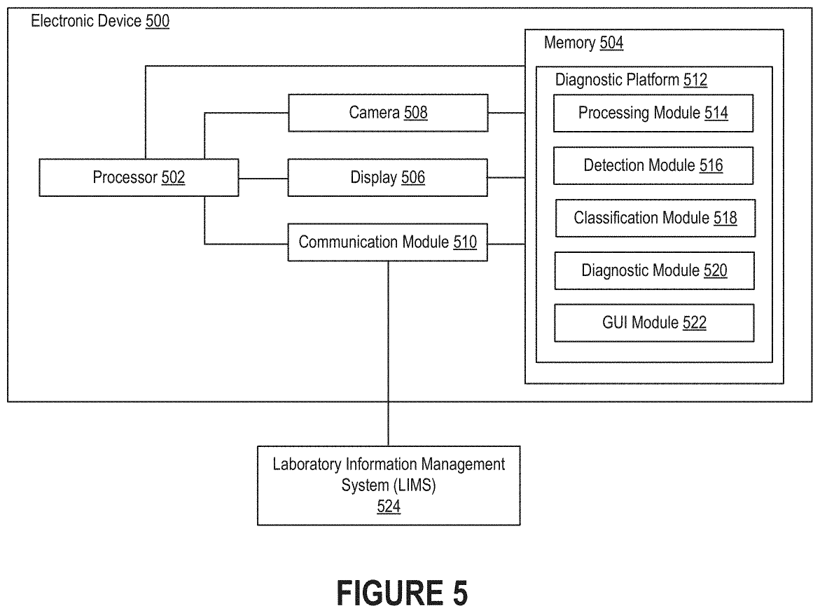

FIG. 5 illustrates an example of an electronic device 500 on which a diagnostic platform 512 may reside. In some embodiments, the diagnostic platform 512 is embodied as a computer program that is executed by the electronic device 500. In other embodiments, the diagnostic platform 512 is embodied as a computer program that is executed by another electronic device (e.g., a computer server) to which the electronic device 500 is communicatively connected. In such embodiments, the electronic device 500 may transmit relevant data, such as images generated by the camera 510, to the other electronic device for processing. Those skilled in the art will recognize that aspects of the diagnostic platform could also be distributed amongst multiple electronic devices.

At a high level, the diagnostic platform 512 may be designed to analyze image data (or simply “images”) related to an individual (also referred to as a “patient” or “subject”) to establish a characteristic of her health. For example, the diagnostic platform 512 may be configured to produce a proposed diagnosis with respect to an ailment, or the diagnostic platform 512 may be configured to generate output(s) that can be used by medical professionals to render more informed diagnoses. As further discussed below, the diagnostic platform 512 is able to accomplish this by discovering and then characterizing objects in the images. Generally, this is done autonomously without involvement from specialists trained to examine images. However, in some embodiments, the processes described below may be performed semi-autonomously in conjunction with a specialist. For example, the diagnostic platform 512 may propose labels indicating how objects in the images should be classified, and an individual may be able to confirm or reject those labels. In some embodiments, the diagnostic platform 512 is configured to produce a report that identifies regions of pixels labelled as representative of abnormal cells using, for example, bounding boxes. In such embodiments, the diagnostic platform 512 may allow the individual to provide input indicative of requests to alter the bounds of a region or the label assigned to a region. Thus, the individual may be able to indicate if the diagnostic platform 512 has improperly defined a region of pixels or improperly labelled a region of pixels. Such an approach may be useful as a way of training the diagnostic platform 512 to further improve its artificial intelligence and machine learning algorithms.

The electronic device 500 can include a processor 502, memory 504, display 506, camera 508, and communication module 510. The communication module 510 may be, for example, wireless communication circuitry designed to establish communication channels with other electronic devices. For example, the communication module 510 may establish a communication channel with a LIMS 524 over which patient-related data can be received and/or transmitted, as discussed above. Examples of wireless communication circuitry include integrated circuits (also referred to as “chips”) configured for Bluetooth, Wi-Fi, NFC, and the like. The processor 502 can have generic characteristics similar to general-purpose processors, or the processor 502 may be an application-specific integrated circuit (ASIC) that provides control functions to the electronic device 500. As shown in FIG. 5, the processor 502 can be coupled to all components of the electronic device 500, either directly or indirectly, for communication purposes.

The memory 504 may be comprised of any suitable type of storage medium, such as static random-access memory (SRAM), dynamic random-access memory (DRAM), electrically erasable programmable read-only memory (EEPROM), flash memory, or registers. In addition to storing instructions that can be executed by the processor 502, the memory 504 can also store data generated by the other components of the electronic device 500. For example, the memory 504 may include images generated by the camera 508 and data produced by the processor 502 (e.g., when executing the modules of the diagnostic platform 512). Note that the memory 504 is merely an abstract representation of a storage environment. The memory 504 could be comprised of actual memory chips or modules.

The communication module 510 can manage communications between the components of the electronic device 500. The communication module 510 can also manage communications with other electronic devices. Examples of electronic devices include mobile phones, tablet computers, personal computers, and network-accessible server systems comprised of computer server(s). For example, in embodiments where the electronic device 500 is associated with a medical professional, the communication module 510 may be communicatively connected to a network-accessible server system managed by a diagnostic service. As another example, the communication module 510 may be communicatively connected to the microscope that includes the slide being imaged. The communication module 510 may initiate wireless communication with the microscope to obtain information related to the examination session, such as the name of the patient, time of the examination session, model of microscope, etc. This information may be used by the diagnostic platform 512 to ensure that each output is associated with the appropriate patient.

As further discussed below, the diagnostic platform 512 may handle several types of data, namely, image data relating to the images captured during microscopic examination of a slide with a blood smear and physiological data relating to the patient whose blood is being examined. The physiological data may be reported by the patient herself. For example, the physiological data may include information provided by the patient before the examination session (e.g., as part of a registration process or enrollment process) or during the examination session (e.g., as part of an intake process). Additionally or alternatively, the physiological data may include information that has been automatically obtained and/or derived on behalf of the patient (e.g., without requiring any input from the patient). As an example, the diagnostic platform 512 may acquire information related to the patient from a network-accessible database managed by a hospital, laboratory, or other medical entity. In FIG. 5, for instance, the diagnostic platform 512 may obtain information related to the patient from the LIMS 524.

For convenience, the diagnostic platform 512 may be referred to as a computer program that resides within the memory 504. However, the diagnostic platform 512 could be comprised of software, firmware, or hardware components implemented in, or accessible to, the electronic device 500. In accordance with embodiments described herein, the diagnostic platform 512 may include a processing module 514, object detection module 516, classification module 518, diagnostic module 520, and graphical user interface (GUI) module 522. These modules can be an integral part of the diagnostic platform 512. Alternatively, these modules can be logically separate from the diagnostic platform 512 but operate “alongside” it.

The processing module 514 can apply one or more operations to images acquired by the diagnostic platform 512. As mentioned above, these images may be generated by the camera 508 as part of a session involving examination of a slide that includes a blood smear. These images may be provided to the diagnostic platform 512 continually (e.g., in real time as those images are generated), periodically (e.g., every 30, 60, or 90 seconds), or in an ad hoc manner (e.g., upon receiving input indicating that the imaging part of the examination session has been completed). Thus, the diagnostic platform 512 may be configured to obtain a series of images generated based on light reflected through the eyepiece of a microscope and then analyze the series of images in real time to detect, classify, and count regions of pixels that are, for example, representative of abnormal cells.

The processing module 514 can process the images into a format suitable for the other modules of the diagnostic platform 512. For example, the processing module 514 may compress the images for storage in the memory 504. As another example, the processing module 514 may alter the hue, saturation, contrast, or some other characteristic of the images. Moreover, upon determining that quality of images has fallen beneath a threshold, the processing module 514 may improve the quality by adjusting color balance, exposure, color temperature, clarity, or sharpness of the camera responsible for generating the images. Accordingly, the processing module 514 may attempt to improve quality in real time as images are being generated and provided for analysis. As another example, the processing module 514 may alter the images to improve the signal-to-noise (SNR) ratio before those images are analyzed by the other modules. As another example, the processing module 514 may be responsible for “stitching” images together to form a composite image (also referred to as a “scan”) that is representative of the slide as a whole. More specifically, upon receiving a series of images, the processing module 514 can examine the coordinates of those images, as well as features occurring along the boundaries, to determine how those images should be aligned with one another. Oftentimes, some images will need to overlap when stitched together, though the degree of overlap may not be consistent. For example, a given image may overlap with its upwardly adjacent neighboring image by 2 millimeters and overlap with its downwardly adjacent neighboring image by 1 millimeter. Stitching the images together, when done appropriately, can help ensure that fewer errors are made when detecting and then counting objects. Stitching the images together may improve the efficiency with which the images can be analyzed by the diagnostic platform 512. While additional computational resources may be needed by the processing module 514 to stitch these images together, the other modules may be able to process the composite image more efficiently than if each individual image were analyzed separately.

The object detection module 516 (or simply “detection module”) may employ AI-driven algorithms to detect objects in the images. Examples of such algorithms include R-CNN, Fast R-CNN, YOLO, and SSD. In some embodiments, the detection module 510 is configured to produce a count of all objects in the images. For example, the object detection module 510 may estimate the number of cells included in a given segment of a blood smear on a slide. While a single instance of the detection module 516 is shown in FIG. 5, multiple object detection algorithms could be applied to the images obtained by the diagnostic platform 512. For example, upon receiving an image, the detection module 516 may apply a model thereto that includes multiple AI-driven algorithms, each of which is designed to detect instances of a different type of object based on an analysis of the pixel data of the image.

Moreover, the detection module 516 may employ AI-driven algorithms to track and count the objects detected in the images. Counting objects, such as cells, can be difficult because the intersection of images can be quite big. Accordingly, objects can easily be counted twice. In order to avoid double counting, the detection module can save the coordinates of bounding boxes corresponding to detected objects, and then when a new bounding box arrives, check it against the saved coordinates to see if it corresponds to a new (i.e., uncounted) cell or an existing (i.e., counted) cell.

The classification module 518 may employ AI-driven algorithms to classify the objects detected by the detection module 516. For example, the classification module 518 may label each cell in an image as either healthy or unhealthy based on its shape, size, and/or color. To accomplish this, the classification module 518 may identify those cells whose characteristics exceed a predetermined threshold in one of these categories. For example, the classification module 518 may automatically label a cell as unhealthy responsive to determining that the cell exceeds a given size. Additionally or alternatively, the classification module 518 may train a model to classify a cell responsive to determining that a given segment of an image is representative of the cell. The classification model may be a neural network that is trained using a supervised machine learning algorithm. For example, the model may consider as input batches of pixels indicative of cells and corresponding labels that have been assigned to those batches of pixels by medical professionals that manually classified the cells.

The diagnostic module 520 may be responsible for generating an output based on the objects as classified by the classification module 518. Examples of outputs include proposed diagnoses, reports, etc. Thus, the diagnostic module 520 may generate a proposed diagnosis for an ailment, such as malaria, based on whether unhealthy cells indicative of the ailment are discovered by the classification module 518. The diagnostic module 520 can also perform analytic process(es) based on the image generated during an examination session, physiological data associated with the corresponding patient, and/or outputs produced by the other modules.

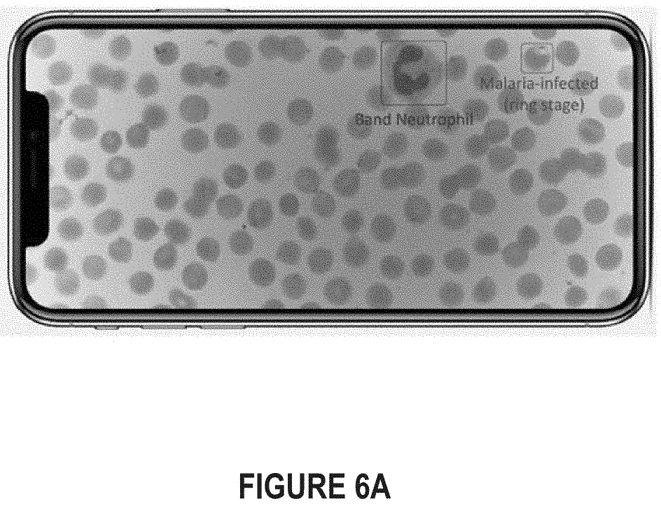

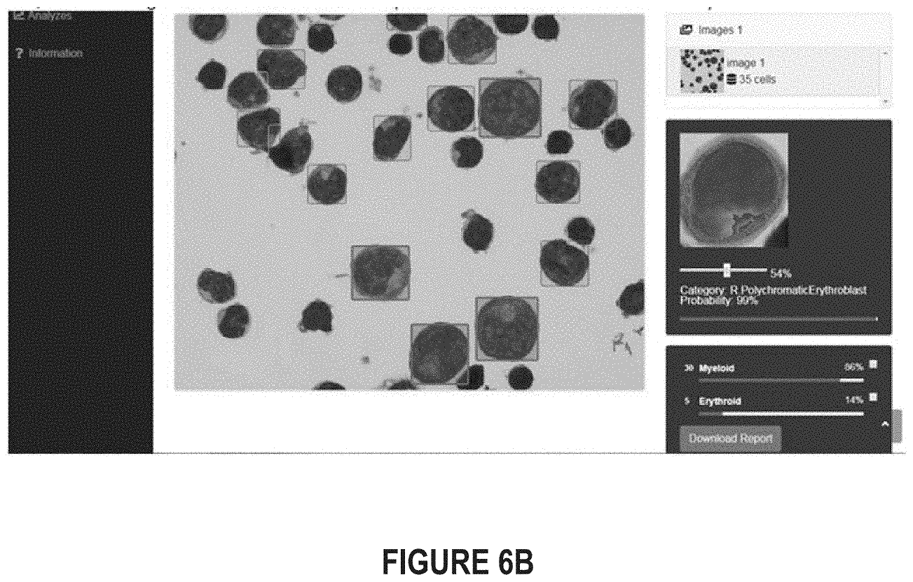

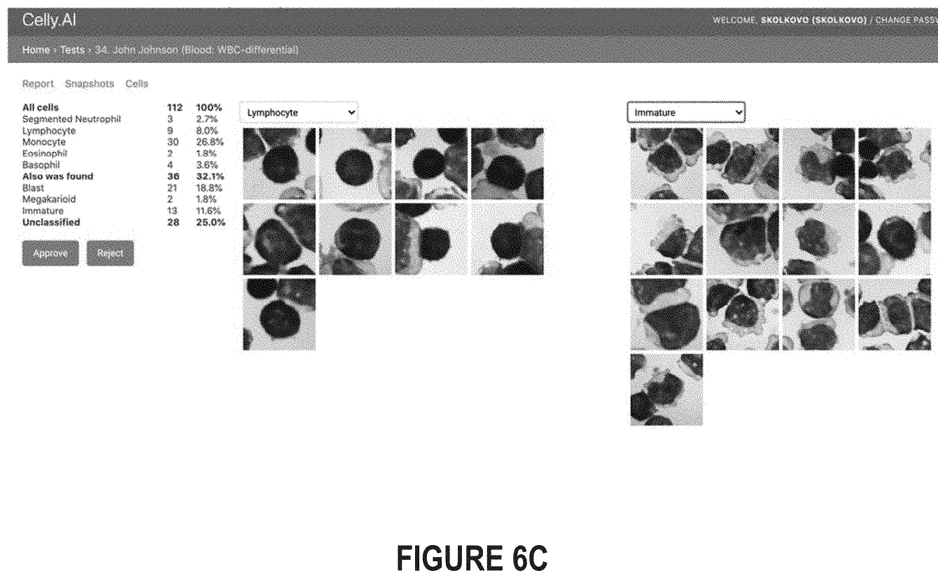

The GUI module 522 can generate the interface(s) through which an individual can interact with the diagnostic platform 512. Example of interfaces are shown in FIGS. 6A-B. In particular, FIG. 6A depicts an example of interface in which the cells deemed abnormal in a blood smear have been visually outlined and labeled as such, while FIG. 6B depicts an example of an interface in which the results of an analysis have been presented. These interfaces may be shown on the display 506 of the electronic device 500, or data representative of the interfaces may be transmitted to another electronic device by the communication module 510 for display. Thus, the individual may be able to readily discover which ailments, if any, the patient under examination is presently suffering from by observing the interfaces generated by the GUI module 522.

FIG. 6C, meanwhile, illustrates how a series of images, rather than a composite image of the entire slide, can be processed by a diagnostic platform and then presented in a report. Initially, the diagnostic platform can process the series of images to detect and then classify the cells contained in those images. Then, the diagnostic platform can present the classified cells in a report. In FIG. 6C, for example, the diagnostic platform has clustered the images into two batches: a first batch determined to contain lymphocyte cells and a second batch determined to contain immature cells. Those skilled in the art will recognize that all images obtained by the diagnostic platform need not necessarily be presented in the report. For example, images that remain unclassified may be assumed to contain only red blood cells, and thus may not be shown. As shown in FIG. 6C, the report generated by the diagnostic platform may be interactive. Thus, the individual that reviews the report may be able to approve or disapprove individual classifications (e.g., by dragging an image from one classification to another), approve or disapprove classification as a whole, view the images corresponding to different classifications (e.g., using the drop-down menu shown in FIG. 6C), etc.

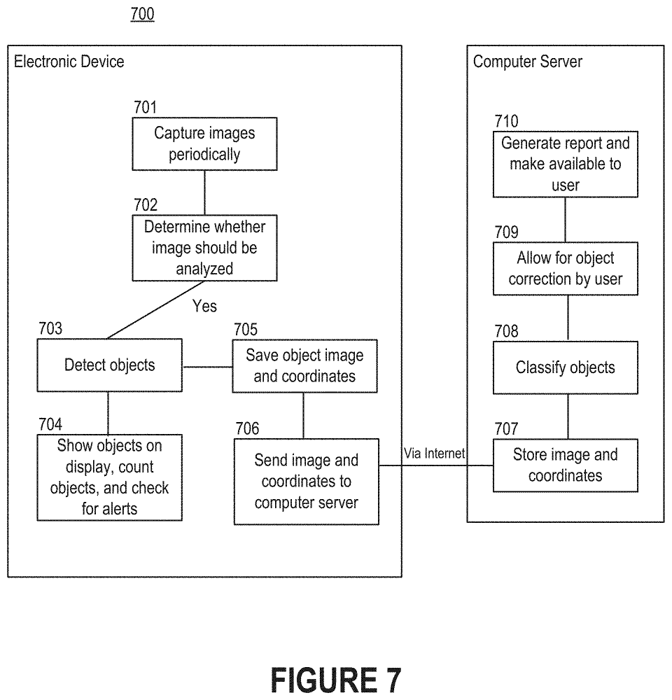

FIG. 7 depicts a flow diagram of a process 700 for analyzing images of cells that are observable through a microscope. As shown in FIG. 7, part of the process 700 may be executed by a computer program executing on an electronic device such as a mobile phone or tablet computer, while another part of the process 700 may be executed by a computer program executing on a computer server. Generally, the computer programs represent portions of a diagnostic platform. Note that the distribution of responsibilities shown in FIG. 7 has been provided solely for the purpose of illustration. In some embodiments, the entire process 700 is performed on the mobile phone. In other embodiments, all analysis is performed on the computer server. That is, all steps of the process 700 may be performed on the computer server except for generating the images.

Initially, the computer program can prompt the electronic device to periodically capture images (step 701). For instance, the images may be generated at a predetermined frequency (e.g., every 20 or 30 milliseconds), or each of the images may be generated responsive to receiving input indicative of a request to capture the image. As an example, an individual may be responsible for relocating the slide across the stage. Each time that the individual relocates the slide (e.g., by shifting the slide in one direction by several millimeters), the computer program or individual may provide input that an image should be generated. For instance, the individual might interact with a digital element shown on the display of the electronic device, or the individual might interact with a physical button of the electronic device.

For each image, the computer program can determine whether the image should be analyzed (step 702). To accomplish this, the computer program may check for the shift between successive images, quality, contrast, etc. For example, the computer program may indicate that the image should be analyzed only if it determines that a certain amount of shift has occurred since the preceding image. As another example, the computer program may indicate that the image should be analyzed only if it determines that the quality (e.g., as measured in resolution, blurriness, etc.) exceeds a certain threshold.

Responsive to determining that a given image should be analyzed, the computer program can perform a series of different actions. For example, the computer program may detect objects, if any, included in the given image by applying an object detection algorithm to its pixel data (step 703). Then, the computer program may show the objects on the display of the electronic device, establish a count of the objects, or perform some other action (step 704). Additionally or alternatively, the computer program may save the object image along with coordinates indicating a position relative to the starting point (step 705). The computer program can then send the images and coordinates to a computer server via, for example, the Internet (step 706). In scenarios where the computer program uploads coordinates with each image, the computer server can use the coordinates to digitally stitch together the images to form a more holistic view of the blood smear. That is, the coordinates may be used to discover how multiple images should be arranged, overlapped, etc.

Upon receiving the image and coordinates, the computer server can store the image in a storage medium (step 707). In some embodiments, the computer server performs more precise object detection. For example, the computer server may apply a more resource-intensive object detection algorithm to confirm that the computer program executing on the electronic device properly detected objects in the image. Alternatively, the electronic device may be responsible for detecting instances of objects in the cells, as described above with reference to step 703, while the computer server may be responsible for classifying those detected objects (step 708). If necessary, the computer server may receive input from a user indicative of corrections to the detected objects (step 709). For example, the user may provide the input by interacting with the image (e.g., by tapping or clicking on the display of the electronic device) to identify objects that were not properly identified by either computer program, to eliminate objects that were incorrectly identified as objects by one of the computer programs, etc. Similarly, the computer server may receive input from a user indicative of corrections to the classifications of those detected objects. In some embodiments, the computer server generates a report that includes the image and information related to the physiological state of a corresponding patient (step 710). Moreover, the computer server may make the image and information related to the physiological state of the corresponding patient available for review.

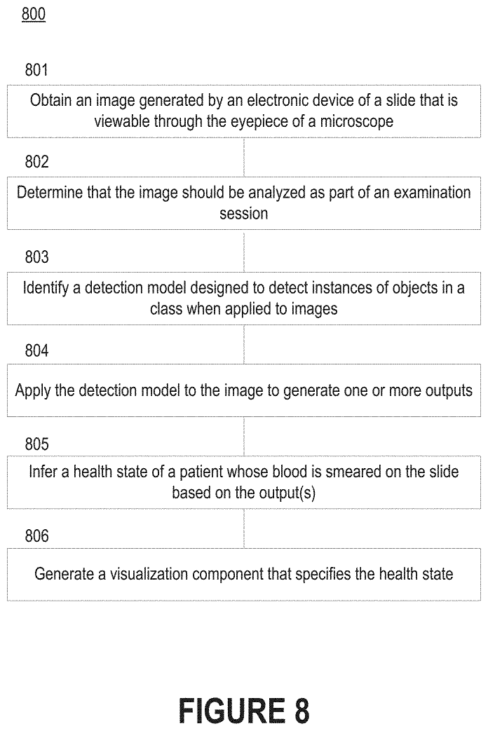

FIG. 8 depicts a flow diagram of a process 800 for inferring health based on automated analysis of images of a blood smear. Initially, a slide with the blood of a patient smeared thereon can be placed on the stage of a microscope by an individual. The individual can also affix an electronic device to the microscope such that the camera of the electronic device is aligned with the eyepiece of the microscope. Thereafter, the individual can cause images of the blood smear to be generated from light reflected through the eyepiece of the microscope. For example, the individual may indicate (e.g., via the display of the electronic device) that the images should be generated by the camera and then analyzed by a diagnostic platform.

Accordingly, a diagnostic platform may obtain an image that was generated by an electronic device of a slide that is viewable through the eyepiece of a microscope (step 801). Upon receiving the image, the diagnostic platform may determine whether the image should be analyzed as part of an examination session (step 801). In some embodiments, the diagnostic platform examines the image to ensure that a characteristic indicative of quality exceeds a predetermined threshold. The characteristic could be, for example, hue, saturation, contrast, SNR ratio, or clarity. In other embodiments, the diagnostic platform examines metadata that accompanies the image to identify a set of coordinates representative of location with respect to the slide and then compares those coordinates to the coordinates of a preceding image. By comparing these sets of coordinates, the diagnostic platform can calculate a shift metric that indicates how much shift occurred between those images. The diagnostic platform may only analyze the image if the shift is determined to exceed a predetermined amount; otherwise, the diagnostic platform may ignore the image due to the amount of overlap with the preceding image.

Thereafter, the diagnostic platform can identify a detection model designed to detect instances of objects in a class when applied to images (step 803). The appropriate detection model may be based on the type of analysis that is desired. For example, if the diagnostic platform receives input indicating that the blood smear should be examined for visual indicators of malaria, the diagnostic platform may identify a detection model designed specifically for malaria. Said another way, the diagnostic platform may identify a detection model comprised of object detection algorithm(s) trained to identify abnormal cells whose presence is indicative of malaria. The diagnostic platform can then apply the detection model to the image to generate one or more outputs (step 804). As shown in FIGS. 6A-B, each output may be a bounding box that defines a perimeter of a region of pixels deemed to be representative of an instance of the object by the detection model. Additionally or alternatively, each output may include a label for the detected instance.

Then, the diagnostic platform can infer a health state of the patient whose blood is smeared on the slide based on the output(s) (step 805). For example, the diagnostic platform may be configured to generate an alert responsive to a determination that a single object indicative of an unhealthy cell was discovered by the detection model, or the diagnostic platform may be configured to generate an alert responsive to a determination that the number of discovered objects indicative of unhealthy cells exceeds a threshold count or percentage of all cells. The alert may be a visual notification in the form of a text message, email message, or report presented for display by the diagnostic platform. In some embodiments, the diagnostic platform is configured to generate a visualization component that specifies the health state as inferred by the diagnostic platform (step 806). The visualization component may include information regarding the patient, blood smear, or examination session. For example, the visualization component may include a proposed diagnosis for an ailment determined by the diagnostic platform based on the output(s) produced by the detection model. The visualization component may be designed for consumption by a medical professional or the patient herself.

Other steps could also be performed in some embodiments. As an example, the diagnostic platform may allow the individual responsible for managing the slide to initiate an analysis mode by specifying a type of analysis to be performed by the diagnostic platform. Examples of types of analysis include complete blood count, bone marrow analysis, malaria detection, and biopsy analysis. The detection model(s) applied by the diagnostic platform may depend on the type(s) of analysis requested by the individual. As another example, the diagnostic platform may enable the individual to input information regarding the blood smear, patient, microscope, or examination session. For instance, the individual may be able to specify a smear identifier or patient identifier through an interface generated by the diagnostic platform, and the diagnostic platform can use this information to ensure that any outputs are associated with the appropriate patient and session.

### Processing System

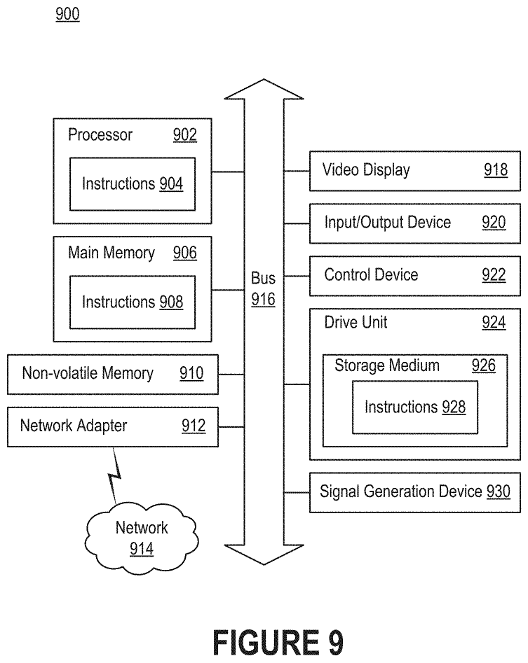

FIG. 9 is a block diagram illustrating an example of a processing system 900 in which at least some operations described herein can be implemented. For example, components of the processing system 900 may be hosted on a computing device that includes a diagnostic platform (e.g., diagnostic platform 402 of FIG. 4 or diagnostic platform 512 of FIG. 5).

The processing system 900 may include a processor 902, main memory 906, non-volatile memory 910, network adapter 912 (e.g., a network interface), video display 918, input/output device 920, control device 922 (e.g., a keyboard, pointing device, or mechanical input such as a button), drive unit 924 that includes a storage medium 926, or signal generation device 930 that are communicatively connected to a bus 916. The bus 916 is illustrated as an abstraction that represents one or more physical buses and/or point-to-point connections that are connected by appropriate bridges, adapters, or controllers. The bus 916, therefore, can include a system bus, Peripheral Component Interconnect (PCI) bus, PCI-Express bus, HyperTransport bus, Industry Standard Architecture (ISA) bus, Small Computer System Interface (SCSI) bus, Universal Serial Bus (USB), Inter-Integrated Circuit (I²C) bus, or bus compliant with Institute of Electrical and Electronics Engineers (IEEE) Standard 1394.

The processing system 900 may share a similar computer processor architecture as that of a computer server, router, desktop computer, tablet computer, mobile phone, video game console, wearable electronic device (e.g., a watch or fitness tracker), network-connected (“smart”) device (e.g., a television or home assistant device), augmented or virtual reality system (e.g., a head-mounted display), or another electronic device capable of executing a set of instructions (sequential or otherwise) that specify action(s) to be taken by the processing system 900.

While the main memory 906, non-volatile memory 910, and storage medium 924 are shown to be a single medium, the terms “storage medium” and “machine-readable medium” should be taken to include a single medium or multiple media that stores one or more sets of instructions 926. The terms “storage medium” and “machine-readable medium” should also be taken to include any medium that is capable of storing, encoding, or carrying a set of instructions for execution by the processing system 900.

In general, the routines executed to implement the embodiments of the present disclosure may be implemented as part of an operating system or a specific application, component, program, object, module, or sequence of instructions (collectively referred to as “computer programs”). The computer programs typically comprise one or more instructions (e.g., instructions 904, 908, 928) set at various times in various memories and storage devices in a computing device. When read and executed by the processor 902, the instructions cause the processing system 900 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile and non-volatile memory devices 910, removable disks, hard disk drives, optical disks (e.g., Compact Disk Read-Only Memory (CD-ROMS) and Digital Versatile Disks (DVDs)), cloud-based storage, and transmission-type media such as digital and analog communication links.

The network adapter 912 enables the processing system 900 to mediate data in a network 914 with an entity that is external to the processing system 900 through any communication protocol supported by the processing system 900 and the external entity. The network adapter 912 can include a network adaptor card, a wireless network interface card, a switch, a protocol converter, a gateway, a bridge, a hub, a receiver, a repeater, or a transceiver that includes an integrated circuit (e.g., enabling communication over Bluetooth or Wi-Fi).

The techniques introduced here can be implemented using software, firmware, hardware, or a combination of such forms. For example, aspects of the present disclosure may be implemented using special-purpose hardwired (i.e., non-programmable) circuitry in the form of application-specific integrated circuits (ASICs), programmable logic devices (PLDs), field-programmable gate arrays (FPGAs), and the like.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A method comprising:
obtaining, by a diagnostic platform, an image generated by an electronic device of a slide viewable through an eyepiece of a microscope;
determining, by the diagnostic platform, that the image should be analyzed as part of an examination session;
identifying, by the diagnostic platform, a detection model designed to detect instances of objects in a class when applied to images;
applying, by the diagnostic platform, the detection model to the image to generate at least one output,
wherein each output is indicative of an instance of an object in the class;
inferring, by the diagnostic platform based on the at least one output, a health state of an individual whose blood is smeared on the slide; and
generating, by the diagnostic platform, a visualization component that specifies the health state.

2. The method of claim 1, wherein the image is one of a series of images obtained by the diagnostic platform.

3. The method of claim 2, wherein said determining comprises:
examining metadata that accompanies the image to identify a set of coordinates representative of location with respect to the slide,
comparing the set of coordinates to coordinates of a preceding image in the series of images, and
confirming, based on said comparing, that a shift exceeding a predetermined amount occurred between the preceding image and the image.

4. The method of claim 1, wherein said determining comprises:
confirming that a characteristic indicative of quality exceeds a predetermined threshold by analyzing the image.

5. The method of claim 4, wherein the characteristic is hue, saturation, contrast, signal-to-noise (SNR) ratio, or clarity.

6. The method of claim 1, wherein the detection model is one of multiple detection models applied to the image by the diagnostic platform.

7. The method of claim 6, wherein each of the multiple detection models applies a different object detection algorithm.

8. The method of claim 1, wherein each of the at least one output is a bounding box that defines a perimeter of a region of pixels representative of the corresponding instance of the object.

9. A system for analyzing images of a slide with a blood smear thereon that is observable through an eyepiece of a microscope, the system comprising:
an optical adapter configured to maintain an electronic device in a predetermined arrangement with respect to the eyepiece of the microscope; and
the electronic device that includes a memory with instructions stored thereon that, when executed by a processor, cause the processor to:
cause a series of images of the slide to be generated by a camera based on light reflected through the eyepiece, and
analyze the series of images in real time to detect, classify, and count regions of pixels representative of abnormal cells in the blood smear.

10. The system of claim 9, wherein the instructions further cause the processor to:
determine quality of each image in the series of images, and
responsive to determining that quality has fallen beneath a threshold,
improve quality by adjusting color balance, exposure, color temperature, clarity, or sharpness of the camera.

11. The system of claim 9, wherein the instructions further cause the processor to:
generate, for each image in the series of images, a shift metric by calculating shift with respect to a preceding image.

12. The system of claim 9, wherein the instructions further cause the processor to:
cause display of an image in which each detected object is visually highlighted.

13. The system of claim 12, wherein the instructions further cause the processor to:
generate an alert responsive to a determination that a detected object is indicative of an unhealthy cell.

14. A method comprising:
placing a slide with a blood smear thereon on a stage of a microscope;
affixing an electronic device to the microscope such that a camera of the electronic device is aligned with an eyepiece of the microscope;
causing the camera of the electronic device to generate a series of images of the blood smear from light reflected through the eyepiece of the microscope; and
initiating analysis of the series of images by a computer program that resides on the electronic device,
wherein the computer program is configured to detect, classify, and count regions of pixels representative of the cells in the series of images.

15. The method of claim 14, further comprising:
relocating the slide on the stage of the microscope on a periodic basis so that each of the series of images is of a different portion of the blood smear.

16. The method of claim 15, further comprising:
prompting generation of each of the series of images by specifying, via the electronic device, when the slide has been relocated.

17. The method of claim 14, further comprising:
initiating an analysis mode by choosing a type of test to be performed,
wherein the type of test is selected from complete blood count, bone marrow analysis, malaria detection/counting, and histopathology analysis.

18. The method of claim 14, further comprising:
inputting, via the electronic device, information regarding the blood smear on the slide,
wherein the information includes a smear identifier or a patient identifier.

19. The method of claim 14, further comprising:
reviewing a report that identifies the regions of pixels labelled as representative of abnormal cells.

20. The method of claim 19, further comprising:
providing, via the electronic device, input indicative of a request to alter a label assigned to a given region of pixels by the computer program.
